(19) United States
(12) Patent Application Publication
(10) Pub. No.: US 2004/0171869 A1
(43) Pub. Date: Sep. 2, 2004

(54) Method for producing mdi, especially 2,4'-mdi

(76) Inventors: Martin Reif, Romerberg (DE); Bernd Bruchmann, Freinsheim (DE); Siegmund Pohl, Diepholz (DE); Carl Bettendorf, Kapellen (BE); Heinz Plaumann, Flat Rock, MI (US); Kai Thiele, Schwarzheide (DE)

Correspondence Address: Basf Corporation, Patent Department, 1609 Biddle Avenue, Wyandotte, MI, 48192, US

(21) Appl. No.: 10/469,847

(22) PCT Filed: Mar. 6, 2002
(86) PCT No.: PCT/EP02/02426

(30) Foreign Application Priority Data
Mar. 8, 2001 (DE) 101 11 337.4

(51) Int. Cl. C07C 263/04

(52) U.S. Cl. 560/347

(57) ABSTRACT

MDI is prepared by a process comprising the following steps:

A) reaction of aniline and formaldehyde in the presence of an acid to give methylene(diphenyldiamines) and polymethylenepolyphenylenepolyamines, the molar ratio of acid to amine being not more than 0.2,

B) reaction of the mixture obtained in step A) with phosgene to give methylene(diphenyl diisocyanates) and polymethylenepolyphenylene polyisocyanates,

C) separation of the mixture obtained in step B) into monomeric MDI and polymeric MDI and

D) separation of the monomeric MDI obtained in step C) by isolating 2,4′-MDI.

The invention relates to a process for the preparation of MDI, comprising the steps A) reaction of aniline and formaldehyde in the presence of an acid as a catalyst to give methylene(diphenyldiamines) and polymethylenepolyphenylenepolyamines, the molar ratio of acid to amine being 0.2 or less, B) reaction of the mixture obtained in step A) with phosgene to give methylene(diphenyl diisocyanates) and polymethylenepolyphenylene polyisocyanates, C) separation of the mixture from step B) into monomeric MDI and PMDI and D) separation of the monomeric MDI obtained in step C) for isolating 2,4′-MDI.

Aromatic iscoyanates are important and versatile raw materials for polyurethane chemistry.

Tolylene diisocyanate (TDI) and MDI are the most important industrial isocyanates here.

The general term “MDI” is used in the technical field and in the context of this application as a general term for methylene(diphenyl diisocyantes) and polymethylenepolyphenylene polyisocyanates. The term methylene(diphenyl diisocyanate) covers the isomers 2,2′-methylene(diphenyl diisocyanate), 2,4′-methylene(diphenyl diisocyanate), (2,4′-MDI) and 4,4′-methylene(diphenyl diisocyanate) (4,4′-MDI). These isomers are referred to together as monomeric MDI. The term polymethylenepolyphenylene polyisocyanates covers polymeric MDI (PMDI), which contains monomeric MDI and higher homologs of monomeric MDI.

Isomers and isomer mixtures differing from MDI are commercially available, and 4,4′-/2,4′-MDI mixtures and PMDI are offered in addition to 4,4′-MDI. Although a large variety of PU polymers having different structures and properties can be prepared using these various isocyanates, there are nevertheless some restrictions: Polymeric MDI is a complex mixture of different MDI oligomers (in general N-nucleus MDI, where N=2->10 and MDI isomers (there are, for example, 7 different 3-nucleus MDI isomers). These products are very useful industrially, but it is not possible to establish individual, defined chemical structures. However, it is essential to establish defined polyurethane structures in order to achieve desired performance characteristics.

Owing to their difunctionality, monmeric MDI types (4,4′-/2,4′-MDI) are more suitable than PMDI for establising defined structures, 4,4′-MDI containing two isocyanate groups of identical reactivity. For this reason, however, specific functionalization at only one isocyanate function is virtually impossible in the case of 4,4′-MDI since a second addition is only insignificantly more unfavorable, and consequently mixtures are usually obtained in addition reactions.

It is therefore desirable to provide a 2-nucleus MDI having two isocyanate groups of different reactivity, in order to obtain novel property profiles. 2,4′-MDI is a molecule which meets these requirements. Here, a reaction in the 4-position is considerably preferred over a reaction in the 2-position for steric reasons, and it is therefore possible to establish a specific structure.

Advantages of pure 2,4′-MDI and 2,4′-MDI-rich MDI mixtures have already been discussed in the prior art.

A. M. Sarpeshkar, P. H. Markusch, R. L. Cline “Designing Polyurethane Elastomers with low Compression Sets” in:

Polyurethanes Conference 2000, Proceedings, Conference Volume, Conference of October 8 to 11, 2000 in Boston, Mass. The advantageous effect of 2,4′-MDI on the compression set is described here for the preparation of PU elastomers having particular properties.

EP-B-0676434 states that the 2,4-TDI/2,6-TDI mixture otherwise used could be replaced, for example, by the use of 2,4′-MDI in PU flexible (molded) foam systems.

EP-B-0431331 discloses the use of 2,4′-MDI in heat-curable 1-component PU systems.

EP-A-0572994 describes the preparation of polyisocyanates having uretdione groups.

DE-A-19904444 and WO 97/02304 describe the synthesis of denrimers and highly branched polyurethanes.

From the above explanations, it is clear that there is a need for 2,4′-MDI, particularly on the part of manufacturers of PU systems. Nevertheless, 2,4′-MDI—although this molecule has long been known—is not commercially available on an industrial scale in the form of the pure isomer. Only 2,4′-MDI and 4,4′-MDI mixtures (e.g. 50/50 Lupranat® MI from BASF) and 2,4′-MDI-rich polymeric MDI prepolymers (e.g. Rubinat® 9483 from Huntsman) are commercially available.

In all industrially relevant production processes, MDI is produced by phosgenation of methylene(diphenyldiamine) (MDA). The synthesis is carried out in a two-stage process. First, aniline is condensed with formaldehyde to give a mixture of oligomeric and isomeric methylene(diphenyldiamines) and polymethylenepolyphenylenepolyamines, i.e. crude MDA. This crude MDA is then reacted in a second step with phosgene in a manner known per se to give a mixture of the corresponding oligomeric and isomeric methylene(diphenyl diisocyanates) and polymethylenepolyphenylene polyisocyanates, i.e. crude MDI. Here, the isomer composition and oligomer composition remain unchanged. In general, a part of the 2-nucleus compound is then separated off in a further process step (for example by distillation or crystallization), polymeric MDI (PMDI) remaining as residue.

From the above explanations, it is clear that, in the production plants of the prior art—with otherwise unchanged product mix, i.e. particularly with otherwise unchanged amounts of 2,4′-/4,4′-MDI mixtures produced—the additional production of pure 2,4′-MDI results in a reduction in the 2,4′-MDI content of the PMDI, since larger amounts of 2,4′-MDI are separated from the crude MDI. However, for reasons relating to performance characteristics, a changed PMDI composition is not desirable since it can adversely affect the performance characteristics of the PU systems produced therefrom. For industrial PMDI production, it is therefore important that the composition of the PDMI remains constant; this explains why 2,4′-MDI has not been commercially available to date.

It is an object of the present invention to provide a process for the preparation of MDI which makes it possible to obtain 2,4′-MDI without the production costs of the MDI process, in particular for the phosgenation step, being substantially increased and without the composition of the product mix of the plant being changed. The product mix of the plant is understood as meaning in particular the composition of the discharged PMDI and the composition and amount of the discharged monomeric MDI mixture.

The necessity of preparing 2,4′-MDI without increasing the production costs of the process should be viewed in the light of the fact that the industrial MDI plants are complicated, continuous plants which have to meet very high requirements with regard to availability and reliability. It is therefore a further object of the present invention to provide a process which can be carried out with little technical complexity even in existing industrial MDI production plants without endangering the availability and reliability of the process.

Although the literature thoroughly describes advantages and processes for the preparation of 2,4′-MDI, a simple, economical process for the preparation of 2,4′-MDI which does not change the composition of the coupled product PMDI is unknown to date.

DE-A-2930411 describes a mixture of 2-nucleus MDI isomers having a high content of 2,2′-MDI and 2,4′-MDI as a solvent for paint stabilizers, antioxidants, flame retardants, etc.

BE 735258 discloses an MDA process using very large amounts of acid, MDA having a very low 2,4′-MDA content being said to be produced. The amounts of acid used are too high for an economical process and in addition little of the desired 2,4′-isomer is obtained.

DE-A-26 31 168 describes a process for the preparation of diisocyanatodiphenylmethane isomers having a set content of chlorine compounds. The preparation of very pure 2-nucleus MDI is carried out by means of a complicated distillation sequence. In the experiments, the preparation of pure 2,4′-MDI is also described. However, the object of the experiment was the preparation of 2-nucleus MDI having a low chlorine content. However, DE-A-26 31 168 does not disclose any information regarding the preparation of larger amounts of 2,4′-MDI without changing the composition of the corresponding PMDI.

U.S. Pat. No. 3,892,634, EP-A-0482490, JP-A-2951782, U.S. Pat. No. 77279C and DE-A-2532722 describe distillation and purification of 2-nucleus MDI with the object of obtaining very pale and very low-chlorine monomeric MDI. The isolation of pure 4,4′-MDI is preferred. Although streams enriched in 2,4′-MDI also occur in these separations or distillations, the preparation of pure 2,4′-MDI was not a subject of these inventions.

U.S. Pat. No. 3,362,979 describes the preparation of MDI mixtures having a high 2,4′-MDI content. For the synthesis of the MDA precursor, however, special solid catalysts are used instead of HCl. The preparation of pure 2,4′-MDI is not described.

RU 2058303 describes the preparation of an MDI mixture having a relatively high 2,4′-MDI content. For this purpose, however, the aniline condensation based on dimethyl or diethyl acetals or formaldehyde is carried out not from pure formaldehyde but in the presence of HCl. The process needs additional starting materials, which contradicts the requirements for a very simple and economical process.

JP 06009539 describes MDI mixtures having a high content of 2,4′-MDI. The fact that this is still liquid at −10° C. and does not crystallize is described as an advantage.

There are also many publications which describe the isolation and purification of 4,4′-MDI. As a result of the process, process streams which have a relatively high content of 2,4′-MDI are always obtained. However, the provision of 2,4′-MDI is not a subject of these inventions. Examples of these include BE 884805, U.S. Pat. No. 470,400, DE-A-21051193, DE-A-2425658, WO 98/25889 and DE-A-2532722.

We have found that this object is achieved, surprisingly, if the amount of acid used as catalyst is reduced at the MDA synthesis stage and the crude MDA obtained is then phosgenated to give crude MDI and is then subjected to a special separation sequence, preferably distillation sequence, for isolating 2,4′-MDI.

The present invention therefore relates to a process for the preparation of MDI, comprising the following steps:

A) reaction of aniline and formaldehyde in the presence of an acid to give methylene(diphenyldiamines) and polymethylenepolyphenylenepolyamines, the molar ratio of acid to amine being not more than 0.2,

B) reaction of the mixture obtained in step A) with phosgene to give methylene(diphenyl diisocyanates) and polymethylenepolyphenylene polyisocyantes,

C) separation of the mixture obtained in step B) into monomeric MDI and polymeric MDI and

D) separation of the monomeric MDI obtained in step C) by isolation of 2,4′-MDI.

For the reaction, described in step A), of aniline with formaldehyde to give methylene(diphenyldiamines) and polymethylenepolyphenylenepolyamines, this mixture being referred to as crude MDA, the starting materials are usually mixed in a suitable mixing apparatus, for example in a mixing pump, a nozzle or a static mixer and reacted in a suitable reaction apparatus, for example in a tubular reactor, a stirred reactor or a reaction column or a combination thereof. The reaction temperature is in general from 20 to 200° C., preferably from 30 to 140° C.

The reaction of step A) is carried out in the presence of an acid as a catalyst, the catalyst preferably being added as a mixture with aniline. Preferred catalysts are mineral acids, for example hydrochloric acid, sulfuric acid and phosphoric acid. Mixtures of acids may also be used. Hydrochloric acid is particularly preferred. If hydrogen chloride is used as the catalyst, it may also be used in gaseous form. In the process of the present invention, the amount of catalyst is chosen so that a molar acid/aniline (Ac/An) ratio of s 0.2, preferably <0.16, particularly preferably less than 0.1, results.

In a preferred embodiment, the reaction of step A) is carried out in an aqueous medium using HCl as the catalyst. Furthermore, the reaction can be carried out in the presence of a solvent. Ether, water and mixtures thereof are particularly suitable. Examples of these are dimethylformamide (DMF), tetrahydrofuran (THF) and diethyl isophthalate (DEIP).

Formaldehyde can be fed to the novel process in the form of monomeric formaldehyde and/or in the form of higher homologs, i.e. poly(oxymethylene)glycols. The molar aniline:formaldehyde ratio is in general from 1.5:1 to 10:1, preferably from 2:1 to 5:1. Furthermore, the reaction can be carried out either continuously or batchwise, in a batch or semibatch process.

In step B), the phosgenation of the crude MDA mixture from step A) is carried out in a manner known per se to a person skilled in the art, for example as described in Chemistry and Technology of Isocyanates by H. Ulrich, John Wiley Publishers, 1996, and the literature cited therein. The solvents used may be all inert aromatic or aliphatic hydrocarbons or halohydrocarbons which are known for the phosgenation process and in which the respective isocyanate is soluble and which are not attacked under the reaction conditions. Aromatic compounds, e.g. monochlorobenzene, o-dichlorobenzene or toluene, are preferably used. The isocyanate which is to be prepared can itself serve as a solvent.

The phosgenation is preferably followed by working-up of the crude isocyanate, in which excess phosgene and solvent are separated off. In a possible embodiment, a physical, e.g. thermal, and/or chemical aftertreatment for removing troublesome byproducts can also follow, as described, for example, in U.S. Pat. No. 3,912,600, DD 288599, U.S. Pat. No. 5,364,958, EP 0133538, JP 06345707, DD 288598, DD 288593, EP 0524507 and EP 0866057. The working-up and aftertreatment are included under step B) for the purposes of this application.

The reaction in step B) gives a mixture of various MDI oligomers and isomers. This mixture is generally known by the term crude MDI. In steps C) and D) of the novel process, separation of the crude MDI is carried out. This can be effected by known methods, for example distillation, solvent extraction, extraction with supercritical media, e.g. supercritical CO₂ or crystallization. Distillation is preferred.

In step C) of the novel process, at least a part of the monomeric MDI is separated off, preferably distilled off, from the crude MDI obtained in step B). The part which remains behind in this separation is generally referred to as polymeric MDI (PMDI). The amount of monomeric MDI separated off depends on the composition which the remaining PMDI is to have and is usually at least 10, preferably from 10 to 70, particularly preferably from 20 to 50,% by weight, based on the amount of crude MDI. The distillate fraction, consisting of monomeric MDI, is then further processed in step D), in particular for the isolation of 2,4′-MDI. The PMDI remaining behind can also be used for polyurethane preparation. Since polyurethanes are systems of isocyanate and polyol which are tailored to one another, it is desirable for the PMDI always to have a constant content of monomeric MDI and PMDI, i.e for different batches not to have different properties.

In step D), the monomeric MDI obtained from step C), substantially containing 2,4′-MDI and 4,4′-MDI, is separated by at least partly separating off 2,4′-MDI. Here, at least 5, preferably from 10 to 80, more preferably from 15 to 70, in particular from 30 to 70,% by weight, based on the total amount of 2,4′-MDI contained in the monomeric MDI obtained from step C), of 2,4′-MDI are separated off. After the separation, a mixture which contains 4,4′-MDI remains behind, or two or more mixtures remain behind, the first containing 4,4′-MDI and the remaining mixtures containing 4,4′-MDI and 2,4′-MDI in identical or different amounts.

In a possible embodiment, the separation is preferably effected in a distillation column, 4,4′-MDI being obtained in the bottom and 2,4′-MDI being obtained as the top product.

In a preferred embodiment, the separation is effected in a distillation column, 4,4′-MDI being obtained in the bottom, 2,4′-MDI being obtained as the top product and a mixture of 2,4′-MDI and 4,4′-MDI being obtained in a side take-off of the column.

In a particularly preferred embodiment, this mixture contains from 30 to 70, preferably about 50,% by weight of 2,4′-MDI and from 3 to 70, preferably about 50,% by weight of 4,4′-MDI. In this embodiment, based on the total amount of monomeric MDI obtained from step C), from 1 to 90, preferably from 40 to 80,% by weight of 4,4′-MDI,

from 1 to 80, preferably from 5 to 30,% by weight of a roughly 50/50 mixture of 2,4′-MDI and 4,4′-MDI and

from 1 to 50, preferably from 2 to 20,% by weight of 2,4′-MDI are usually obtained.

In a further embodiment, the mixture C) can be separated into pure 4,4′-MDI and a mixture of 2,4′-MDI and 4,4′-MDI in a first distillation column. This mixture of 2,4′-MDI and 4,4′-MDI can be subjected to a further distillation in which the 2,4′-MDI is isolated. 4,4′-MDI remains behind as residue.

In a further embodiment, the separation of the isomers as obtained from step C) can also be carried out by crystallization, as described, for example, in DE 2322574, GB 1417087, U.S. Pat. No. 4,014,914, BE 215525, DE 2606364 or DE 19645659, or by solvent extraction, as described, for example, in DD 118618, U.S. Pat. No. 4,876,380, U.S. Pat. No. 4,871,460 or DE 4200236.

In a preferred embodiment, the separation according to step D) is effected in a distillation column. It is furthermore preferable if separation step C) is carried out by distillation and separation step D) by crystallization and/or solvent extraction.

It is also possible for separation step C) to be carried out by crystallization and/or solvent extraction and separation step D) by distillation.

Compared with the prior art processes, the novel process has the following advantages: 2,4′-MDI is provided as an isocyanate in relatively large amounts without the composition of the product mix of the plant, in particular the composition of the simultaneously obtained coupled product PDMI being substantially changed and without a radical change in the existing processes for the preparation of isocyanates by phosgenation being necessary. Moreover, the novel process makes it possible to reduce the required amount of acid (catalyst) in the MDA process. Consequently, firstly less sodium hydroxide solution is required for subsequent neutralization of the acid, i.e. costs for the necessary alkali can be reduced, and secondly there is a smaller salt load in the wastewater, i.e. less environmental pollution.

A constant PMDI composition is important for the preparation of polyurethane systems. The novel process makes it possible to establish constant compositions in a controlled manner with respect to the oligomer and isomer distribution of the PMDI.

Thus, for example in rigid foam applications, a high monomeric MDI content is desirable in order to achieve good flow behavior.

However, a low and constant 2,4′-MDI content is also decisive for achieving thorough curing. In addition, owing to the low 2,4′-MDI content, good crosslinking in the foam and hence high compressive strength are obtained. The PMDI obtained in step C) therefore preferably has a 2,4′-MDI content of from 1 to 6, more preferably from 1.5 to 4, in particular from 2 to 4,% by weight, so that, for example, sensitive rigid foam systems can be produced in a advantageous, reproducible and reliable manner.

The examples which follow illustrate the invention.

### COMPARATIVE EXAMPLE

In a continuous process, aniline is reacted with aqueous formaldehyde solution in an A/F ratio of 2.2. Aqueous, concentrated hydrochloric acid is added as a catalyst. The ratio of acid to aniline is 0.25. The reaction mixture thus obtained is worked up in a manner known per se, i.e. the crude mixture is first neutralized with sodium hydroxide solution and then washed salt-free with water. Thereafter, first water and then unconverted aniline are distilled off. The water-free crude MDA thus obtained (5.95 t/h) is reacted with 8.5 t/h of phosgene in chlorobenzene as a process solvent at 120° C. in stirred kettles to give isocyanate. The mixture leaving the phosgenation is freed from phosgene and chlorobenzene and subjected to thermal aftertreatment according to the prior art. About 7.5 t/h of crude MDI are obtained.

25% (1.86 t/h) of monomeric MDI are then separated by distillation from the crude MDI obtained, so that 5.64 t/h of polymeric MDI remain behind. The isocyanate characteristics of this product were determined and are shown in table 1. Table 2 shows the oligomer composition. The 2-nucleus MDI is subjected to a further distillation in a column, 1.6 t/h of pure 4,4′-MDI being obtained in the bottom of the column. In addition, 0.26 t/h of a 50/50 mixture of 2,4′-MDI and 4,4′-MDI is obtained. Pure 2,4′-MDI not obtained in this process.

### EXAMPLE 1

### According to the Novel Process, Ac/An=0.14

In a continuous industrial process, aniline is reacted with aqueous formaldehyde solution in an A/F ratio of 2.3. Aqueous, concentrated hydrochloric acid is added as a catalyst. The acid-to-aniline ratio is 0.14. The reaction mixture thus obtained is worked up in a manner known per se, i.e. the crude mixture is first neutralized with sodium hydroxide solution and then washed salt-free with water. Thereafter, first water and then unconverted aniline are distilled off. The water-free crude MDA thus obtained (5.95 t/h) is reacted with 8.5 t/h of phosgene in chlorobenzene as a process solvent at 120° C. in stirred kettles to give the isocyanate. The mixture leaving the phosgenation is freed from phosgene and chlorobenzene and subjected to a thermal aftertreatment according to the prior art. About 7.5 t/h of crude MDI are obtained.

25% (1.86 t/h) of 2-nucleus MDI are then separated off by distillation from the crude MDI obtained, so that 5.64 t/h of polymeric MDI remain behind. The isocyanate characteristics of this product were determined, and these characteristics are shown in table 1. Table 2 shows the oligomer composition. The 2-nucleus MDI is subjected to a further distillation in a column, 1.42 t/h of pure 4,4′-MDI being obtained in the bottom of the column. In addition, 0.44 t/h of a 50/50 mixture of 2,4′-MDI and 4,4′-MDI is obtained. 0.26 t/h of this mixture is filled directly to a storage tank and sold. The remaining 0.18 t/h is subjected to a further distillation, about 0.09 t/h of pure 2,4′-MDI being produced. In addition, about 0.09 t/h of pure 4,4′-MDI is obtained and is mixed with the 1.42 t/h 4,4′-MDI stream obtained above so that 1.51 t/h of 4,4′-MDI are obtained altogether.

### EXAMPLE 2

### According to the Novel Process, Ac/An=0.-09

In a continuous process, aniline is reacted with aqueous formaldehyde solution in an A/F ratio of 2.3. Aqueous, concentrated hydrochloric acid is added as a catalyst. The ratio of acid to aniline is 0.09. The reaction mixture thus obtained is worked up in a manner known per se, i.e. the crude mixture is first neutralized with sodium hydroxide solution and then washed salt-free with water. Thereafter, first water and then unconverted aniline are distilled off. The water-free crude MDA thus obtained (1 kg/h) is reacted with 1.4 kg/h of phosgene in chlorobenzene as a process solvent at 120° C. in a stirred kettle to give isocyanate. The mixture leaving the phosgenation is freed from phosgene and chlorobenzene and subjected to thermal aftertreatment according to the prior art. About 1.26 kg/h of crude MDI are obtained.

25% (316 g/h) of 2-nucleus MDI are then separated by distillation from the crude MDI obtained, so that 944 g/h of polymeric MDI remain behind. The isocyanate characteristics of this product were determined and are shown in table 1. Table 2 shows the oligomer composition. The 2-nucleus MDI is subjected to a further distillation in a column, 204 g/h of pure 4,4′-MDI being obtained in the bottom of the column. In addition, 112 g/h of a 50/50 mixture of 2,4′-MDI and 4,4′-MDI is obtained. 44 g/h of this mixture are filled directly into a storage container. The remaining 68 g/h are subjected to a further distillation, about 34 g/h of pure 2,4′-MDI being produced. The isocyanate characteristics of the 2,4′-MDI are shown in table 3. In addition, about 34 g/h of pure 4,4′-MDI are obtained and are mixed with the 204 g/h 4,4′-MDI stream obtained above so that 238 g/h of 4,4′-MDI are obtained altogether.

TABLE 1
Characterization of the PMDI

| | NCO | Viskosität | 2,4″-MDI | TC | EHC | DHC |
|---|---|---|---|---|---|---|
| | [%] | [mPas] | [%] | [ppm] | [ppm] | [ppm] |
| Comparison | 31.65 | 180 | 2.15 | 1400 | 85 | 1050 |
| Example 1 | 31.85 | 180 | 2.15 | 1350 | 85 | 1000 |
| Example 2 | 31.85 | 180 | 2.2 | 1500 | 100 | 1100 |

EHC Acidity (easily hydrolyzable chlorine), measured according to ASTM D 4667-87
DHC Hydrolyzable chlorine (difficultly hydrolyzable chlorine), measured according to ASTM D 4663-87.
TC Total chlorine content, determined by X-ray fluorescence analysis
NC Isocyanate content, measured according to DIN 53185
Viscosity Determined according to DIN 51550

TABLE 2
Oligomer composition of the PMDI

| | | | Σ 2- | Σ 3- | Σ 4- |
|---|---|---|---|---|---|
| | 2,4″-MDI | 4,4′-MDI | nucleus | nucleus | nucleus |
| | [%] | [%] | MDI [%] | MDI [%] | MDI [%] |
| Comparison | 2.15 | 36.7 | 38.88 | 29.03 | 8.50 |
| Example 1 | 2.15 | 35.9 | 38.10 | 29.38 | 8.33 |
| Example 2 | 2.2 | 35.6 | 37.8 | 29.68 | 8.4 |

TABLE 3
Characteristic of the 2,4′-MDI

| | |
|---|---|
| TC [ppm] | 87 |
| NCO [%] | 33.5 |
| 2,4″-MDI content* [%] | 99.5 |
| 4,4″-MDI content* [%] | 0.23 |

*Gas chromatographic determination

TABLE 4
Mass balance, flow rates

| | | | | | 50/50 |
|---|---|---|---|---|---|
| | Crude | | | | 2,4′-/4,4′-MDI |
| | MDI | PMDI | 4,4′-MDI | 2,4′-MDI | mixture |
| Com- | 7.5 t/h | 5.64 t/h | 1.6 t/h | 0 t/h | 0.26 t/h |
| parison | (100%) | (75.2%) | (21.3%) | | (3.5%) |
| Example 1 | 7.5 t/h | 5.64 t/h | 1.51 t/h | 0.09 t/h | 0.26 t/h |
| | (100%) | (75.2%) | (20.1%) | (1.2%) | (3.5%) |
| Example 2 | 1260 g/h | 944 g/h | 238 g/h | 34 g/h | 44 g/h |
| | (100%) | (74.9%) | (18.9%) | (2.7%) | (3.5%) |

We claim:

1. A process for the preparation of MDI, comprising the following steps:
A) reaction of aniline and formaldehyde in the presence of an acid to give methylene(diphenylediamines) and polymethylenepolyphenylenepolyamines, the molar ratio of acid to amine being not more than 0.2,
B) reaction of the mixture obtained in step A) with phosgene to give methylene(diphenyl diisocyanates) and polymethylenepolyphenylene polyisocyanates,
C) separation of the mixture obained in step B) into monomeric MDI and polymeric MDI and
D) separation of the monomeric MDI obtained in step C) with isolation of 2,4′-MDI.

2. A process as claimed in claim 1, wherein the separation steps C) and D) are carried out by distillation.

3. A process as claimed in claim 1, wherein the separation steps C) and D) are carried out by crystallization and/or solvent extraction.

4. A process as claimed in claim 1, wherein separation step C) is carried out by distillation and separation step D) by crystallization and/or solvent extraction.

5. A process as claimed in any of claims 1 to 4, wherein the polymeric MDI obtained in step C) has a 2,4′-MDI content of from 1 to 6% by weight.

6. A process as claimed in any of claims 1 to 5, wherein, in step D), the monomeric MDI obtained in step C) is separated into the components
D1) 4,4′-MDI and
D3) 2,4′-MDI.

7. A process as claimed in any of claims 1 to 5, wherein, in step D), the monomeric MDI obtained in step C) is separated into the components
D1) 4,4′-MDI,
D2) a mixture of 2,4′-MDI and 4,4′-MDI and
D3) 2,4′-MDI.

8. A process as claimed in claim 7, wherein the mixture D2) consists of about 50% by weight each of 2,4′-MDI and 4,4′-MDI.
